(19) United States
(12) Plant Patent Application Publication
(10) Pub. No.: US 2014/0082777 P1
(43) Pub. Date: Mar. 20, 2014

(54) "Actinidia chinensis planchon" plant

(50) ;

(71) Applicants: Universita' Degli Studi Di Udine, Udine (IT); Alma Mater Studiorum-Universita' Di Bologna, Bologna (IT)

(72) Inventors: Guglielmo Costa, Bologna (IT); Raffaele Testolin, Udine (IT); Guido Cipriani, Udine (IT)

(73) Assignees: Universita' Degli Studi Di Udine, Udine (IT); Alma Mater Studiorum-Universita' Di Bologna, Bologna (IT)

(21) Appl. No.: 13/987,767

(22) Filed: Aug. 29, 2013

(30) Foreign Application Priority Data
Aug. 29, 2012 (EP) 20121668

(51) Int. Cl. A01H 5/00

(52) U.S. Cl. PLT/156

(57) ABSTRACT

A new and distinct Actinidia chinensis Planchon cultivar named AC1536 is disclosed, characterized by distinctive large, oblong fruit, and an earlier time of maturity for harvesting.

### LATIN OF THE GENUS AND SPECIES

Actinidia chinensis Planchon

### VARIETY DENOMINATION

AC1536

### BACKGROUND OF THE INVENTION

The new cultivar is a product of a planned breeding program in Italy under the direction of the Guglielmo COSTA, Raffaele TESTOLIN, and Guido CIPRIANI, all Italian citizens. The seed parent is the seedling variety referred to as Actinidia chinensis Planchon A0172. The pollen parent is the seedling variety referred to as Actinidia chinensis Planchon A0134.16.

Fruit of the new variety was first evaluated in year 2003 with favorable results. After the first evaluation, semi-hardwood cuttings were made of AC1536 and were grafted onto Hayward, Actinidia deliciosa, as rootstocks. Evaluation, asexual propagation, and grafting all first took place at the inventor's commercial nursery at University of Bologna, Italy. Subsequent evaluations of the variety have shown the characteristics to be true to type.

### SUMMARY OF THE INVENTION

The cultivar AC1536 has not been observed under all possible environmental conditions. The phenotype may vary somewhat with variations in environment such as temperature, day length, and light intensity, without, however, any variance in genotype.

The following traits have been repeatedly observed and are determined to be the unique characteristics of AC1536. These characteristics in combination distinguish AC1536 as a new and distinct Actinidia chinensis Planchon cultivar: 1. Distinctive size and shape of fruit. 2. Early time of maturity for harvesting.

### COMPARISON TO PARENT

Plants of the new cultivar AC1536 are similar to plants of the seed parent, Actinidia chinensis Planchon A0172 in most horticultural characteristics. However, plants of the new cultivar AC1536 produce fruit several weeks earlier than Hayward and Hort16A. Average fruit size of AC1536 is 105 grams compared to other A. chinensis cultivar which average fruit weight ranged from 75 to 100 g.

Plants of the new cultivar AC1536 are similar to plants of the pollen parent, Actinidia chinensis A0134.16 in some horticultural characteristics. However, as the sex expression of A0134.16 is male, plants of A0134.16 produce only male flowers, whereas the sex expression of AC1536 is female, and plants produce female flowers that in turn develop into large fruit.

### COMMERCIAL COMPARISON

The new variety is best compared to the commercial varieties Soreli, Hort16A (U.S. Plant Pat. No. 11,066), and Jintao. AC1536 is similar to Soreli in many horticultural characteristics, but AC1536 bears oblong fruit, and bears the fruit earlier in the season than Soreli.

AC1536 is similar to Hort16A in many horticultural characteristics, but AC1536 bears fruit that does not have the protruding beak found in fruit of Hort16A. Moreover, fruits of AC1536 are ripe earlier in the season and the flowering takes place later in the season with respect to Hort16A.

AC1536 is similar to Jintao in many horticultural characteristics, but AC1536 bears larger fruit than Jintao, and bears the fruit earlier in the season. Fruits of AC1536 are less prone to develop cracks.

### BRIEF DESCRIPTION OF THE PHOTOGRAPH

The accompanying photographs in FIGS. 1 and 2 illustrate in full color typical buds on plants of AC1536.

FIG. 3 illustrates in full color typical foliage of AC1536.

FIGS. 4-8 illustrate in full color typical flowers of AC1536.

FIGS. 9-12 illustrate in full color typical foliage, hanging fruit, and stems on plants of AC1536.

FIG. 13 illustrates in full color typical fruit harvested from AC1536.

FIG. 14 illustrates in full color a side-by-side comparison of typical fruit harvested from AC1536 with typical fruit harvested from Hort16A and from Jintao.

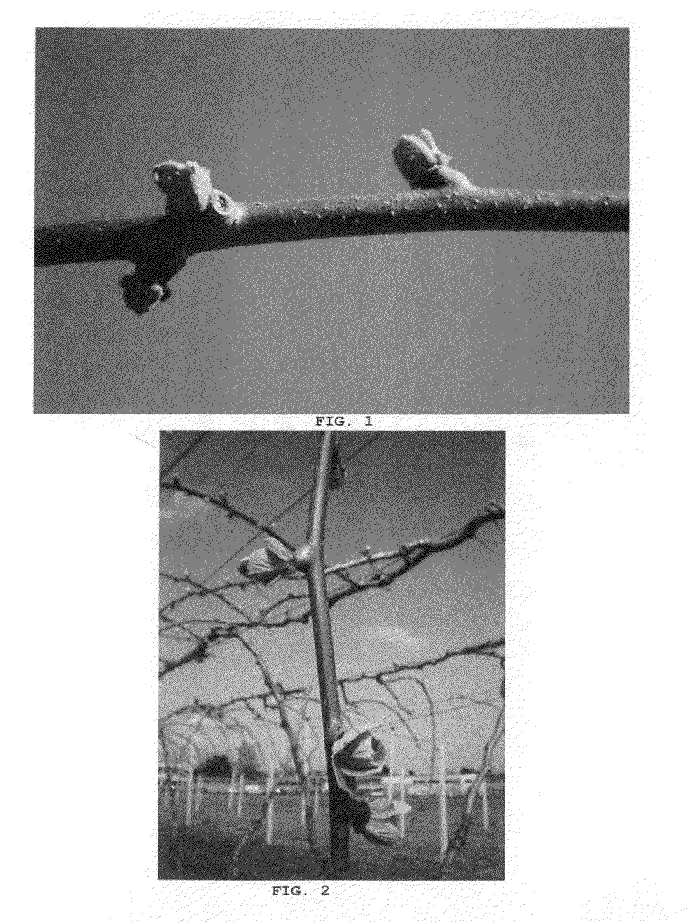

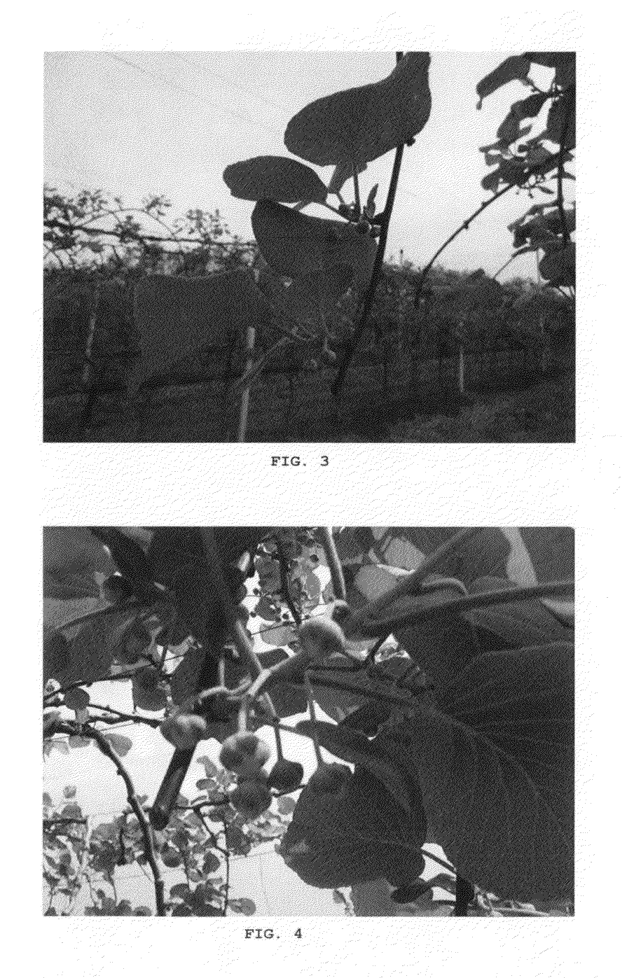

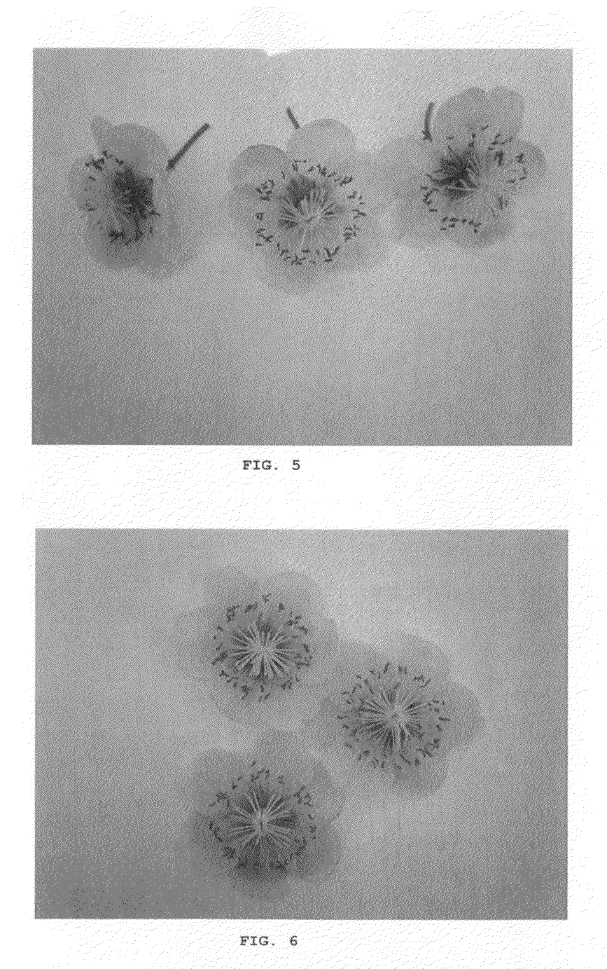

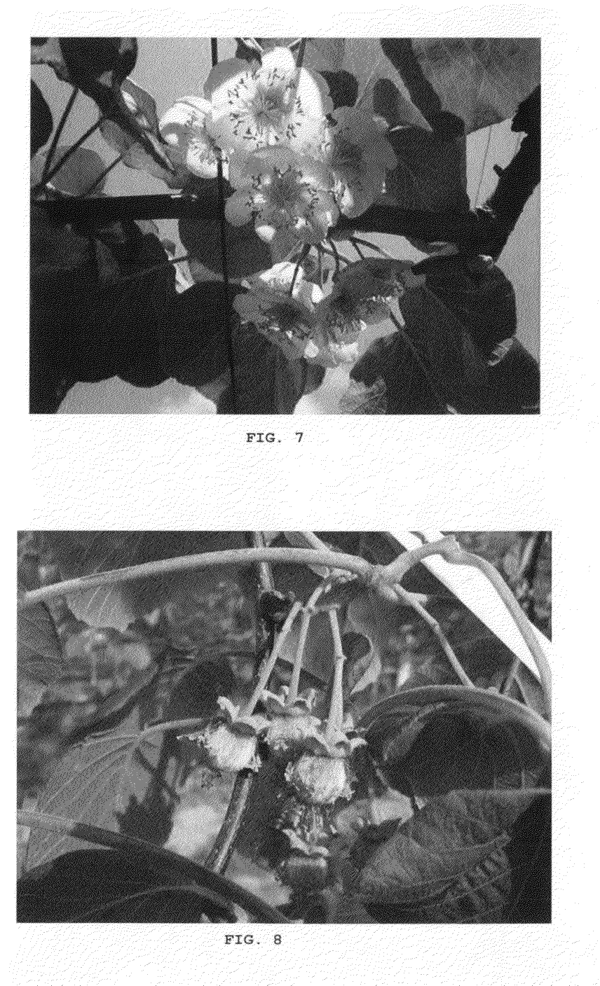

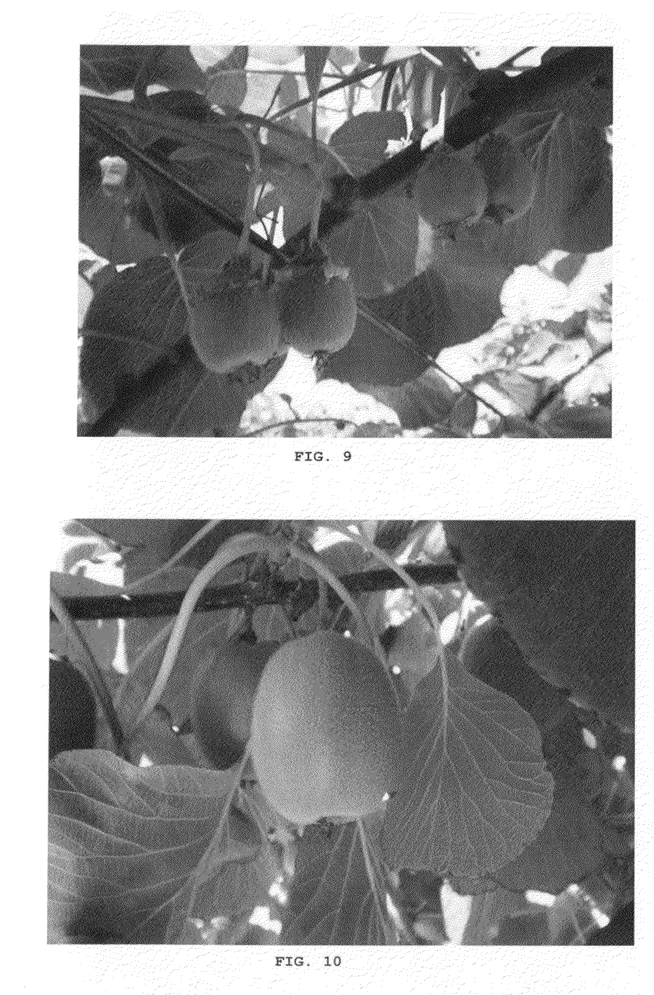

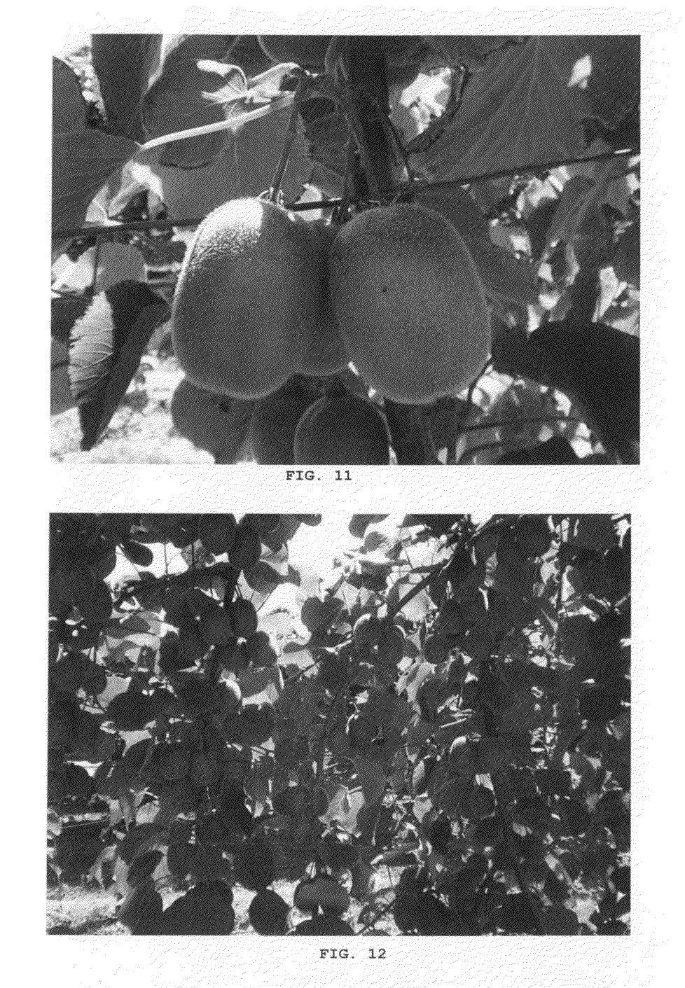

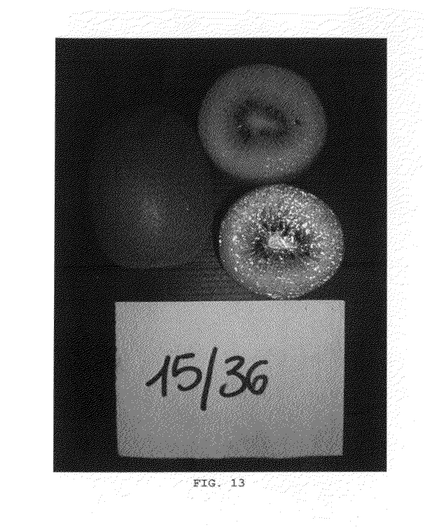

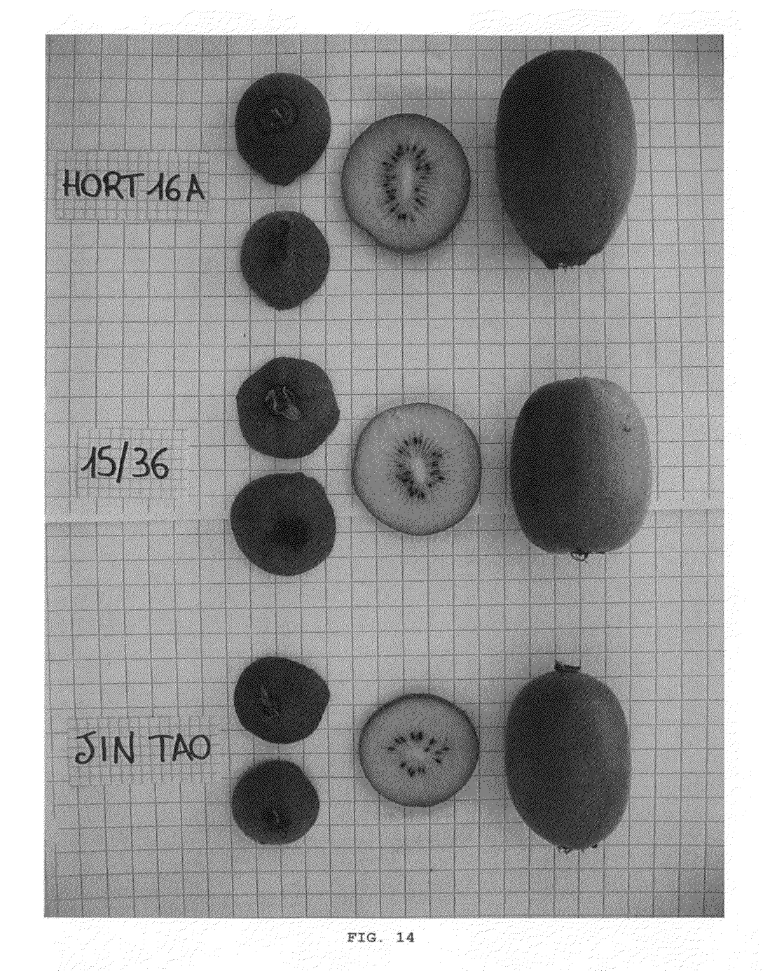

All photographs are taken of plants that are approximately 4/5 years old. The photographs were taken using conventional techniques and although colors may appear different from actual colors due to light reflectance the colors are as accurate as possible by conventional photographic techniques.

### DETAILED BOTANICAL DESCRIPTION

The following observations and measurements describe AC1536 plants grown outdoors under commercial trial conditions in Bologna, Italy. The growing temperature ranged from a minimum of −15° C. in winter (January) up to 40-42° in summer (July-August). Light differences between day and night during the summer days. Annual rainfall is approximately 600 mm per year. Measurements and numerical values represent an average of 10 typical plant types. Botanical classification: Actinidia chinensis Planchon AC1536.

PROPAGATION AC1536 can be successfully grafted onto rootstocks of Actnidia deliciosa and chinensis Planchon.
Plant:
   Age of the plant described.—Approximately 10 years.
   Sex expression.—Female.
   Ploidy.—tetra Diploid.
   Vigor.—Weak-medium.
   Young shoot color.—green.
   Young shoot texture.—Density of hair medium.
   Cane diameter.—Average approximately 1.5 cm.
   Cane texture.—presence of hair.
   Trunk diameter.—Average approximately 7 cm in 10 years old plant.
   Stem lenticels.—very small number and size of lenticels, irregularly spaced and sized. Oblong in shape, color grey-green.
Foliage:
   Leaf.—length. — Approximately 2.2-14 cm; Average length. — Approximately 8 cm.
   Width.—Approximately 16-18.7 cm; Average width. — Approximately 17 cm.
   Shape of blade.—Very broadly obovate.
   Apex.—acuminate.
   Base.—touching each other.
   Attachment.—petioled.
   Margin.—Ciliate.
   Texture of top surface.—Slightly puckered. Non-pubescent.
   Texture of under side.—pubescent, slightly velveted.
   Color.—green. Mature foliage under side: green.
   Petiole.—Length: Average length approximately 59 mm; Average diameter approximately 5 mm.
   Pubescence.—None.
   Color.—Upper surface red, lower surface orange-red.
Flower:
   Flowers per inflorescence.—Normally 1.
   Bud color.—brown.
   Bud break.—End of March.
   First flower.—Mid-May.
   Diameter.—Very large; average approximately 5.3 cm.
   Petal quantity.—average 6 per flower. Average length 31 mm and average width 23 mm approximately. Apex curvature: weakly expressed.
   Petals overlapping.—Yes.
   Color.—White.
   Filament color.—White.
   Another color.—Yellow-orange.
   Attitude of styles.—Semi-erect.
   Style color.—White.
   Style quantity.—Many more than 20.
   Hair on ovary.—Dense.
   Color of ovary.—White.
   Number of sepals.—Average 6.
   Color of sepals.—Green.
   Sepal width.—Average approximately 0.7 cm.
   Sepal length.—Average approximately 0.9 cm.
   Sepal texture.—smooth.
   Peduncle.—Length. — Average approximately 4 cm; Color. — Yellow-green; Texture. — Smooth.
Fruit:
   Color outer pericarp.—Yellow. Color inner pericarp. — Yellow. Core color. — White. Brix at consumption. — Average approximately 16.0. Brix at harvest. — Average approximately 7.50% at the beginning of September up to average approximately 16° Brix at 15 September. Average weight. — approximately 105 grams. Minimum weight. — approximately 90 grams. Maximum weight. — approximately 130 grams.
   Length.—Avg. approximately 7.2 cm.
   Width.—Avg. approximately 5.2 cm.
   Core diameter (maximum).—approximately 1.2 cm. Core diameter (minimum). — approximately 0.7 cm.
   Fruit peduncle length.—Average approximately 5.0 cm. Fruit peduncle width. — Average approximately 0.4 cm; General shape. — Oblong.
   Shoulder shape.—Squared.
   Calyx ring.—Present. Calyx ring expression. — Weakly-expressed.
   Skin color at harvest.—Yellow-brown.
   Hair on fruit skin.—Downy.
   Hair adherence to skin.—Moderate.
   Skin adherence to flesh at maturity.—Moderate.
   Fruit core shape.—Transversely elliptic.
   Core-woody spike.—Absent.
   Lenticels on fruit.—Minute, moderate in quantity, colored yellow-green.
   Mature seed color.—Black. Dried seed. — Brown. Usual harvest time 1^{st} decade (1^{st}-10^{th}) of September. Overall cropping quantity. — 30 to 50 tons per hectare.
Other characteristics:
   Storage life.—Storage life is a minimum of 3 months at 2 degrees C. Disease/pest resistance. — Neither resistance nor susceptibility to pathogens and pests common to Actinidia chinensis Planchon have been observed. Temperature tolerance: Tolerates low temperatures to approximately −5 degrees C. and higher without negative effects, tolerates high temperatures to approximately 35 degrees C. without negative effects.

What is claimed is:

1. A new and distinct cultivar of the Actinidia chinensis Planchon plant named AC1536 as herein illustrated and described.
